(19) United States
(12) Patent Application Publication
(10) Pub. No.: US 2025/0143982 A1
(43) Pub. Date: May 8, 2025

(54) ORAL CARE COMPOSITIONS

(71) Applicant: Colgate-Palmolive Company, New York, NY (US)

(72) Inventors: Jean D. DENIS, Union, NJ (US); Robert Walter D'AMBROGIO, Princeton, NJ (US); Guofeng XU, Plainsboro, NJ (US)

(73) Assignee: Colgate-Palmolive Company, New York, NY (US)

(21) Appl. No.: 18/941,633

(22) Filed: Nov. 8, 2024

Related U.S. Application Data
(60) Provisional application No. 63/547,757, filed on Nov. 8, 2023.

(51) Int. Cl. A61K 8/21; A61K 8/81; A61Q 11/00

(52) U.S. Cl. CPC A61K 8/21; A61K 8/8147; A61Q 11/00

(57) ABSTRACT

Oral care compositions and, particularly, oral care compositions having enhanced stannous ion stability are disclosed herein. In accordance with at least one aspect, provided is an oral care composition comprising: a stannous source; and a cross-linked polyacrylic acid and/or a salt thereof, wherein the personal care composition has a weight ratio of the stannous source to the cross-linked polyacrylic acid and/or a salt thereof of about 4:1 to about 1:4.

### CROSS-REFERENCE TO RELATED APPLICATIONS

This application claims the benefit of priority from U.S. Patent Application No. 63/547,757, filed Nov. 8, 2023, titled Oral Care Compositions, the content of which is hereby incorporated herein in its entirety for all purposes.

### BACKGROUND

Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries tooth decay or tooth demineralization caused by acid produced from the bacterial degradation of fermentable sugar.

Oral care compositions which contain stannous sources exhibit excellent clinical benefits, particularly in the reduction of gingivitis. Stannous sources, such as stannous fluoride and stannous chloride, are well known for use in clinical dentistry with a history of therapeutic benefits over forty years. However, until recently, its popularity has been limited by its instability in aqueous solutions. The instability of stannous salts in water is primarily due to the reactivity of the stannous ion (Sn2+). Stannous salts readily hydrolyze at a pH above 4, resulting in precipitation from solution. It has traditionally been thought that this formation of insoluble stannous salts results in a loss of therapeutic properties.

One common way to overcome the stability problems associated with stannous ions is to limit the amount of water in the composition to very low levels, or to use a dual phase system. Both of these solutions to the stannous ion problem have drawbacks. Low water oral care compositions can be difficult to formulate with desired rheological properties, and dual-phase compositions are considerably more expensive to manufacture and package. Thus, it is preferable to formulate a high-water composition which uses an alternative means to maintain stable efficacious stannous ion concentrations.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions and, e.g., oral care compositions having enhanced stannous ion stability. In accordance with at least one aspect, provided is an oral care composition comprising: a stannous source; and a cross-linked polyacrylic acid and/or a salt thereof, wherein the personal care composition has a weight ratio of the stannous source to the cross-linked polyacrylic acid and/or a salt thereof of about 4:1 to about 1:4.

In accordance with further aspects, provided is an oral care composition comprising: a stannous source; from about 0.2 to about 4 wt. % of a cross-linked polyacrylic acid and/or a salt thereof, a polyphosphate and/or a salt thereof, a polyol; and from about 10 to about 40 wt. % of water, wherein all weight percentages are based on the total weight of the oral care composition.

In accordance with additional aspects, a method is provided for preparing an oral care composition, the method comprising cross-linking a polyacrylic acid and/or salt thereof to form a cross-linked polyacrylic acid and/or salt thereof, and chelating a stannous source with the cross-linked polyacrylic acid and/or salt thereof.

A list of non-limiting example embodiments is provided below:

In accordance with an embodiment 1, provided is an oral care composition comprising: a stannous source; and a cross-linked polyacrylic acid and/or a salt thereof, wherein the personal care composition has a weight ratio of the stannous source to the cross-linked polyacrylic acid and/or a salt thereof of about 4:1 to about 1:4.

In accordance with an embodiment 2, provided is an oral care composition according to claim 1 further comprising: about 10 wt. % of water or more, based on the total weight of the oral care composition.

In accordance with an embodiment 3, provided is an oral care composition according to embodiment 1 or embodiment 2, wherein the water is present in an amount of about 15 wt. % or more, based on the total weight of the oral care composition.

In accordance with an embodiment 4, provided is an oral care composition according to any foregoing embodiment, wherein the cross-linked polyacrylic acid and/or the salt thereof is a branched polyacrylic acid and/or a salt thereof.

In accordance with an embodiment 5, provided is an oral care composition according to any foregoing embodiment, wherein the cross-linked polyacrylic acid and/or the salt thereof is cross-linked with allyl sucrose and/or allyl pentaerythritol.

In accordance with an embodiment 6, provided is an oral care composition according to any foregoing embodiment, wherein the cross-linked polyacrylic acid and/or the salt thereof contains from about 56 to about 68% w/w carboxylic acid groups.

In accordance with an embodiment 7, provided is an oral care composition according to any foregoing embodiment, wherein the cross-linked polyacrylic acid and/or the salt thereof has an average molecular weight of 7×10⁵ to 4×10⁹ Da.

In accordance with an embodiment 8, provided is an oral care composition according to any foregoing embodiment, wherein the cross-linked polyacrylic acid and/or the salt thereof is present in an amount from about 0.2 to about 4 wt. %, based on the total weight of the oral care composition.

In accordance with an embodiment 9, provided is an oral care composition according to any foregoing embodiment, wherein the oral care composition comprises about 2 wt. % or less of natural gums and/or polysaccharides.

In accordance with an embodiment 10, provided is an oral care composition according to any foregoing embodiment, wherein the stannous source is selected from stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, and a combination of two or more thereof.

In accordance with an embodiment 11, provided is an oral care composition according to any foregoing embodiment, wherein the stannous source comprises stannous fluoride.

In accordance with an embodiment 12, provided is an oral care composition according to any foregoing embodiment further comprising: a polyphosphate, optionally, in an amount from about 0.1 to about 7 wt. %, based on the total weight of the oral care composition.

In accordance with an embodiment 13, provided is an oral care composition according to embodiment 12, wherein the polyphosphate is an alkali metal polyphosphate.

In accordance with an embodiment 14, provided is an oral care composition according to embodiment 13, wherein the alkali metal polyphosphate is selected from a pyrophosphate, a tripolyphosphate, a tetraphosphate, a hexametaphosphate, and a combination of two or more thereof.

In accordance with an embodiment 15, provided is an oral care composition according to one of embodiment 12 to embodiment 14, wherein the polyphosphate is selected from sodium pyrophosphate, potassium pyrophosphate, sodium tripolyphosphate, potassium tripolyphosphate, and a combination of two or more thereof.

In accordance with an embodiment 16, provided is an oral care composition according to one of embodiment 12 to embodiment 15, wherein the polyphosphate comprises tetrasodium pyrophosphate, tetrapotassium pyrophosphate, sodium tripolyphosphate, tetrapolyphosphate, sodium trimetaphosphate, sodium hexametaphosphate, or a combination of two or more thereof.

In accordance with an embodiment 17, provided is an oral care composition according to any foregoing embodiment further comprising: a basic amino acid, optionally, in an amount from about 0.1 to about 10 wt. %, based on the total weight of the oral care composition.

In accordance with an embodiment 18, provided is an oral care composition according to embodiment 17, wherein the basic amino acid comprises arginine, lysine, histidine, or a combination thereof.

In accordance with an embodiment 19, provided is an oral care composition according to any foregoing embodiment further comprising: a hydroxide, optionally present in an amount from about 0.1 to about 4 wt. %.

In accordance with an embodiment 20, provided is an oral care composition according to any foregoing embodiment further comprising: a bicarbonate, a carbonate, or a combination thereof, optionally, present in an amount from about 0.1 to about 4 wt. %.

In accordance with an embodiment 21, provided is an oral care composition according to any foregoing embodiment further comprising: a potassium source, optionally, in an amount from about 0.1 to about 6 wt. %, based on the total weight of the oral care composition.

In accordance with an embodiment 22, provided is an oral care composition according to embodiment 21, wherein the potassium source comprises potassium nitrate, potassium citrate, or a combination thereof.

In accordance with an embodiment 23, provided is an oral care composition according to embodiment 21 or embodiment 22, wherein the potassium source comprises potassium nitrate.

In accordance with an embodiment 24, provided is an oral care composition according to any foregoing embodiment further comprising: a polyol, optionally, in an amount from about 30 to about 68 wt. %, based on the total weight of the oral care composition.

In accordance with an embodiment 25, provided is an oral care composition according to embodiment 24, wherein the polyol is present in an amount from about 35 to about 58 wt. %, based on the total weight of the oral care composition.

In accordance with an embodiment 26, provided is an oral care composition according to embodiment 24 or embodiment 25, wherein the polyol comprises glycerin, inositol, maltitol, mannitol, sorbitol, xylitol, propylene glycol, polypropylene glycol (PPG), polyethylene glycol (PEG), a block copolymer of PPG and PEG, a saccharide, or a combination of two or more thereof.

In accordance with an embodiment 27, provided is an oral care composition according to one of embodiment 24 to embodiment 26, wherein the polyol comprises two or more polyols.

In accordance with an embodiment 28, provided is an oral care composition according to embodiment 27, wherein the two or more polyols comprises sorbitol.

In accordance with an embodiment 29, provided is an oral care composition according to any foregoing embodiment further comprising: an organic acid buffer system, optionally, in an amount from about 0.2 to about 4 wt. %, based on the total weight of the oral care composition.

In accordance with an embodiment 30, provided is an oral care composition according to embodiment 29, wherein the organic acid buffer system comprising citric acid and trisodium citrate in a weight ratio of citric acid to sodium citrate of about 1:3 to about 1:8.

In accordance with an embodiment 31, provided is an oral care composition according to any foregoing embodiment, wherein the oral care composition has a pH of about 5 or more, e.g., as measured as a 10 wt. % aqueous solution.

In accordance with an embodiment 32, provided is an oral care composition according to foregoing embodiment, wherein the oral care composition has a reduction of less than about 25% of soluble Tin II when maintained at a temperature of 60° C. for 2 weeks.

In accordance with an embodiment 33, provided is an oral care composition according to any foregoing embodiment, wherein the oral care composition has a reduction of less than about 25% of soluble Tin II when maintained at a temperature of 40° C. and at a relative humidity of 75% for 13 weeks.

In accordance with an embodiment 34, provided is an oral care composition according to any foregoing embodiment, wherein the oral care composition has a reduction of less than about 10% of the total amount of soluble tin when maintained at a temperature of 40° C. and a relative humidity of 75% for 13 weeks.

In accordance with an embodiment 35, provided is an oral care composition comprising: a stannous source; from about 0.2 to about 4 wt. % of a cross-linked polyacrylic acid and/or a salt thereof, a polyphosphate and/or a salt thereof, a polyol; and from about 10 to about 40 wt. % of water, wherein the oral care composition has a pH of about 5 or more and all weight percentages are based on the total weight of the oral care composition.

In accordance with an embodiment 36, a method is provided for preparing an oral care composition comprising: cross-linking the polyacrylic acid and/or the salt thereof to form a cross-linked polyacrylic acid and/or salt thereof; and chelating a stannous source with the cross-linked polyacrylic acid and/or the salt thereof.

In accordance with an embodiment 37, provided is an oral care composition according to embodiment 36 further comprising: adding at least one of a potassium source, a nitrate source, a fluoride source, a polyol, an abrasive, a surfactant, a thickening agent other than a polyacrylic acid and/or salt thereof, a sweetener, a flavoring agent, a colorant, water, or a combination of two or more thereof.

In accordance with an embodiment 38, provided is an oral care composition comprising a stannous source; a polymer system comprising a cross-linked polyacrylic acid, a phosphate/acrylate copolymer, a salt thereof, or a combination thereof, and a polyphosphate and/or a salt thereof, wherein the oral care composition has a pH of about 5 or more and all weight percentages are based on the total weight of the oral care composition.

In accordance with an embodiment 38, provided is an oral care composition comprising a stannous source; a polymer system comprising a cross-linked polyacrylic acid, a phosphate/acrylate copolymer, a salt thereof, or a combination thereof; a nitrate source; a polyphosphate and/or a salt thereof, and about 10 wt. % or more of water, wherein the oral care composition has a pH of about 5 or more and all weight percentages are based on the total weight of the oral care composition.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “° ” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 1000 C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a fluoride source and a stannous source. If a particular oral care composition recites both a fluoride source and a stannous source, stannous fluoride will serve only as either a fluoride source or a stannous source—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions and, e.g., oral care compositions comprising a stannous source and a cross-linked polyacrylic acid and/or a salt thereof. The inventors discovered that oral care compositions disclosed herein can advantageously stabilize the stannous ions in compositions having ingredients that often react with, precipitate, or oxidize such stannous ions. For instance, certain embodiments of the oral care composition achieve stabilized stannous ion while having about 10 wt. % or more or about 15 wt. % or more of water, based on the total weight of the oral care composition. It was further discovered that the oral care compositions according to certain embodiments disclosed herein achieve the stabilized stannous ions while having a pH of about 5 or more.

Preferably, the oral care compositions have an enhanced stannous ion stability, such that the oral care composition has a reduction of less than about 25%, preferably less than about 20%, preferably less than about 18%, of soluble Tin II when maintained at a temperature of 60° C. for 2 weeks. Additionally or alternatively, the oral care compositions may have a reduction of less than about 25%, preferably less than about 23%, preferably less than about 21%, of soluble Tin II when maintained at a temperature of 40° C. and at a relative humidity of 75% for 13 weeks.

In certain preferred embodiments, the oral care composition has an enhanced stannous ion stability, such that the oral care composition has a reduction of less than about 10%, preferably less than about 9%, less than about 8%, or less than 7%, of the total amount of soluble tin when maintained at a temperature of 40° C. and a relative humidity of 75% for 13 weeks.

In accordance with at least one aspect, provided is an oral care composition comprising: a stannous source; and a cross-linked polyacrylic acid and/or a salt thereof, wherein the personal care composition has a weight ratio of the stannous source to the cross-linked polyacrylic acid and/or a salt thereof of about 4:1 to about 1:4.

In accordance with further aspects, provided is an oral care composition comprising: a stannous source; from about 0.2 to about 4 wt. % of a cross-linked polyacrylic acid and/or a salt thereof, a polyphosphate and/or a salt thereof, a polyol; and from about 10 to about 40 wt. % of water, wherein all weight percentages are based on the total weight of the oral care composition.

In accordance with additional aspects, a method is provided for preparing an oral care composition, the method comprising cross-linking a polyacrylic acid and/or salt thereof to form a cross-linked polyacrylic acid and/or salt thereof, and chelating a stannous source with the cross-linked polyacrylic acid and/or salt thereof.

In some embodiments, oral care composition has a pH of about 5 or more. For instance, the oral care composition may have a pH of about 5 to about 10, about 5 to about 9, about 5 to about 8, about 5 to about 7, about 5 to about 6; from about 6 to about 10, about 6 to about 9, about 6 to about 8, or about 6 to about 7.

Additionally or alternatively, the oral care compositions may have a reduced amount thickening agents other than a cross-linked polyacrylic acid, a phosphate/acrylate copolymer and/or salt(s) thereof, such as polysaccharides and/or natural gums. For example, in some preferred embodiments, the oral care composition may have about 4 wt. % or less, about 3 wt. % or less, about 2 wt. % or less, about 1 wt. % or less, about 0.5 wt. % or less, or about 0.1 wt. % or less, based on the weight of the oral care composition, of polysaccharides. Additionally or alternatively, the oral care composition may in some preferred embodiments have about 4 wt. % or less, about 3 wt. % or less, about 2 wt. % or less, about 1 wt. % or less, about 0.5 wt. % or less, or about 0.1 wt. % or less, based on the weight of the oral care composition, of natural gums. Furthermore, in certain preferred embodiments, the oral care composition may have about 4 wt. % or less, about 3 wt. % or less, about 2 wt. % or less, about 1 wt. % or less, about 0.5 wt. % or less, or about 0.1 wt. % or less, based on the weight of the oral care composition, of thickening agents other than a cross-linked polyacrylic acid, a phosphate/acrylate copolymer and/or salt(s) thereof.

The oral care composition may, in certain preferred embodiments, comprise thickening agents other than a cross-linked polyacrylic acid, a phosphate/acrylate copolymer and/or salt(s) thereof in an amount from about 0.1 to about 2.5 wt. % about 0.3 to about 2.5 wt. %, about 0.6 to about 2 wt. %, about 1 to about 2.5 wt. %; from about 0.1 to about 2 wt. % about 0.3 to about 2 wt. %, about 0.6 to about 2 wt. %, about 1 to about 2 wt. %; from about 0.1 to about 1.5 wt. % about 0.3 to about 1.5 wt. %, about 0.6 to about 1.5 wt. %, or any range or subrange thereof from about 0.1 to about 1 wt. % about 0.3 to about 1 wt. %, about 0.6 to about 1 wt. %, or any range or subrange thereof, based on the total weight of the oral care composition. As mentioned above, in certain preferred embodiments, the oral care composition may have a reduced amount of thickening agents other than polyacrylic acid and/or a salt thereof. For instance, the oral care composition may comprise thickening agents other than polyacrylic acid and/or a salt thereof in an amount from about 0.1 to about 2.5 wt. % about 0.3 to about 2.5 wt. %, about 0.6 to about 2 wt. %, about 1 to about 2.5 wt. %; from about 0.1 to about 2 wt. % about 0.3 to about 2 wt. %, about 0.6 to about 2 wt. %, about 1 to about 2 wt. %; from about 0.1 to about 1.5 wt. % about 0.3 to about 1.5 wt. %, about 0.6 to about 1.5 wt. %, or any range or subrange thereof from about 0.1 to about 1 wt. % about 0.3 to about 1 wt. %, about 0.6 to about 1 wt. %, or any range or subrange thereof, based on the total weight of the oral care composition.

The oral care compositions may be in the form of dentifrice, toothpaste, mouthwash, gels, varnishes, leave-on products (e.g., in the form of a gel, varnish, etc.), and/or the like. Suitable components, such as those listed below, may be included or excluded from the formulations for the oral care compositions depending on the specific combination of other ingredients and the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions typically comprise a stannous source, preferably in an effective amount. The stannous source may comprise or consist of a stannous ion source. The stannous source (e.g., stannous ion source) may be present in the oral care composition in an amount from about 0.1 to about 5 wt. %, based on the total weight of the oral care composition. For example, the amount of stannous ion present in the oral care composition may be from about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition. In at least one embodiment, the oral care composition includes about 0.4 to about 0.5 wt. % of stannous source (e.g., stannous ion source), such as about 0.45 wt. % of stannous source.

The stannous source (e.g., stannous ion source) may comprise, or in some cases consist of, stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, or a combination of two or more thereof. For example, the stannous source may comprise stannous fluoride, stannous chloride, stannous acetate, stannous pyrophosphate, and a combination of two or more thereof. The stannous source may be selected from stannous fluoride, stannous chloride, stannous acetate, and a combination of two or more thereof. In at least one embodiment, the stannous source comprises stannous fluoride.

The oral care composition may include two or more, three or more, four or more, five or more, or six or more of stannous source (e.g., stannous ion source). For instance, oral care composition may comprise 2 to 7, 2 to 6, 2 to 5, 2 to 5, or 2 to 4; 3 to 7, 3 to 6, 2 to 5, or 3 to 5 stannous ion sources. In some embodiments, the oral care composition comprises stannous fluoride and stannous pyrophosphate. Additionally or alternatively, the oral care composition may comprise stannous fluoride and stannous chloride.

The oral care compositions typically comprise one or more cross-linked polyacrylic acid, a phosphate/acrylate copolymer, salt(s) thereof or a combination thereof. In at least one preferable embodiment, the oral care compositions comprises one or more cross-linked polyacrylic acid and/or a salt thereof. The total amount of cross-linked acrylic acid and/or a salt thereof in the oral care composition may, in some cases, be from about 0.1 to about 10 wt. %, based on the total weight of the oral care composition. For example, the total amount of cross-linked acrylic acid and/or a salt thereof present in the oral care composition may be from about 0.1 to about 7 wt. %, about 0.1 to about 5 wt. %, about 0.1 to about 3 wt. %, about 0.1 to about 1 wt. %; from about 0.5 to about 10 wt. %, about 0.5 to about 7 wt. %, about 0.5 to about 5 wt. %, about 0.5 to about 3 wt. %, about 0.5 to about 1.5 wt. %; from about 0.1 to about 10 wt. %, about 0.1 to about 7 wt. %, about 0.1 to about 5 wt. %, about 0.1 to about 3 wt. %, about 0.1 to about 1 wt. %; from about 1 to about 10 wt. %, about 1 to about 7 wt. %, about 1 to about 5 wt. %, about 1 to about 3 wt. %, about 1 to about 2 wt. %; from about 2 to about 10 wt. %, about 2 to about 7 wt. %, about 2 to about 5 wt. %; from about 4 to about 10 wt. %, about 4 to about 7 wt. %; from about 6 to about 10 wt. %, about 6 to about 8 wt. %, or any range or subrange thereof, based on the total weight of the oral care composition.

Preferably, the one or more cross-linked acrylic acid and/or a salt thereof may be present in an amount from about 0.2 to about 4 wt. %, based on the total weight of the oral care composition. In some instances, the one or more cross-linked acrylic acid and/or a salt may be present in an amount from about 0.2 to about 3 wt. %, about 0.2 to about 2.5 wt. %, about 0.2 to about 2 wt. %, about 0.2 to about 1.5 wt. %, about 0.2 to about 1.25 wt. %, about 0.2 to about 1 wt. %, about 0.2 to about 0.75 wt. %, about 0.2 to about 0.5 wt. %; from about 0.35 to about 4 wt. %, about 0.35 to about 3 wt. %, about 0.35 to about 2.5 wt. %, about 0.35 to about 2 wt. %, about 0.35 to about 1.5 wt. %, about 0.35 to about 1.25 wt. %, about 0.35 to about 1 wt. %, about 0.35 to about 0.75 wt. %, about 0.35 to about 0.5 wt. %; from about 0.5 to about 4 wt. %, about 0.5 to about 3 wt. %, about 0.5 to about 2.5 wt. %, about 0.5 to about 2 wt. %, about 0.5 to about 1.5 wt. %, about 0.5 to about 1.25 wt. %, about 0.5 to about 1 wt. %, about 0.5 to about 0.75 wt. %; from about 0.75 to about 4 wt. %, about 0.75 to about 3 wt. %, about 0.75 to about 2.5 wt. %, about 0.75 to about 2 wt. %, about 0.75 to about 1.5 wt. %, about 0.75 to about 1.25 wt. %, about 0.75 to about 1 wt. %; from about 1 to about 4 wt. %, about 1 to about 3 wt. %, about 1 to about 2.5 wt. %, about 1 to about 2 wt. %, about 1 to about 1.5 wt. %, about 1 to about 1.25 wt. %; from about 1.5 to about 4 wt. %, about 1.5 to about 3 wt. %, about 1.5 to about 2.5 wt. %, about 1.5 to about 2 wt. %; from about 2 to about 4 wt. %, about 2 to about 3 wt. %, about 2 to about 2.5 wt. %; from about 2.5 to about 4 wt. %, about 2.5 to about 3 wt. %, about 3 to about 4 wt. %, or any range or subrange thereof, based on the total weight of the oral care composition.

The cross-linked polyacrylic acid and/or a salt thereof may be selected from branched polyacrylic acids and/or salts thereof. In some instances, the polyacrylic acid and/or a salt thereof consists of branched polyacrylic acids and/or salts thereof. In further instances, however, the oral care composition includes at least one linear polyacrylic acid and/or a salt thereof.

The cross-linked polyacrylic acid and/or a salt thereof may be a carbomer. An exemplary structure of a monomer of a carbomer is shown below.

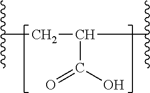

The cross-linked polyacrylic acid and/or a salt thereof may be a high-molecular-weight polyacrylic acid. Preferably, the cross-linked polyacrylic acid and/or a salt thereof is cross-linked with allyl sucrose and/or allyl pentaerythritol. Additionally or alternatively, the cross-linked polyacrylic acid and/or a salt thereof may contain from about 56 to about 68% w/w carboxylic acid groups. For instance, the cross-linked polyacrylic acid and/or a salt thereof may contain from about 56 to about 65% w/w, about 56 to about 62% w/w, about 56 to about 59% w/w; from about 59 to about 65% w/w, about 59 to about 62% w/w, or about 61 to about 65% w/w of carboxylic acid groups. The molecular weight of carbomer may be from 7×10⁵ to 4×10⁹ Da. In some cases, the molecular weight of carbomer is from 7×10⁵ to 1×10⁸ Da, 7×10⁵ to 4×10⁷ Da, 7×10⁵ to 4×10⁶ Da; from 1×10⁶ to 1×10⁸ Da, 1×10⁶ to 4×10⁷ Da; from 1×10⁷ to 1×10⁸ Da, 7×10⁷ to 1×10⁸ Da, or any range or subrange thereof. The carbomer may be selected from Carbopols. For instance, the personal care composition may include, or in some cases exclude, a polyacrylic acid and/or a salt thereof selected from Carbopol 2984, Carbopol 910, Carbopol 934P, Carbopol Ultrez 10, Carbopol 940, Carbopol 941, Carbopol 956, Carbopol 971P, Carbopol 974P, Carbopol 981, Carbopol 2020, and a combination of two or more thereof. Additionally or alternatively, the cross-linked polyacrylic acid and/or a salt thereof may be selected from carbomers. In certain preferred embodiments, the carbomer is C₁₀₋₃₀ alkyl acrylate cross-polymer. In at least one embodiment, the carbomer comprises or consists of Carbopol ETD NF 2020.

The oral care compositions may preferably be formulated to have stannous ion source and cross-linked polyacrylic acid and/or a salt thereof present in a weight ratio of the stannous ion source to the cross-linked polyacrylic acid and/or a salt thereof of about 4:1 to about 1:4. In some instances, the oral care composition has a weight ratio of the stannous ion source to the cross-linked polyacrylic acid and/or a salt thereof of about 4:1 to about 1:4, about 4:1 to about 1:3, about 4:1 to about 1:2, about 4:1 to about 1:1; from about 3:1 to about 1:4, about 3:1 to about 1:3, about 3:1 to about 1:2, about 3:1 to about 1:1; from about 2:1 to about 1:4, about 2:1 to about 1:3, about 2:1 to about 1:2, about 2:1 to about 1:1; from about 1:1 to about 1:4, about 1:1 to about 1:3, about 1:1 to about 1:2, or any range or subrange thereof. In at least one embodiment, the oral care composition has a weight ratio of the stannous ion source to the cross-linked polyacrylic acid and/or a salt thereof of about 1:1.1.

The polyacrylic acid and/or a salt thereof can be neutralized using a cross-linking agent selected from inorganic or organic base, such as alkalimetal-, ammonium-, or (alkylol) amine compound, such as sodium- and potassium hydroxide or- carbonate, ammonia, triethanolamine and the like. However, the inventors unexpectedly discovered that certain crosslinking agents in combination with certain polyacrylic acid and/or a salt thereof, stannous sources, and in particular ratios disclosed herein may achieve enhanced benefits. Thus, one or more of the foregoing cross-linking agent for neutralizing the polyacrylic acid and/or a salt thereof may be included or in some cases excluded from certain embodiments of the oral care composition.

Without being limited to any particular theory, the inventors discovered certain crosslinking agents in combination with certain polyacrylic acid and/or a salt thereof, stannous sources, and in particular ratios disclosed herein promote an enhancement in the stannous stability of certain oral care compositions disclosed herein. The crosslinking agents, as used herein, are typically adapted to crosslink the polyacrylic acid and/or a salt thereof. The crosslinking agents, in some cases, may comprise, or in some cases consist of, those selected from polyphosphates, basic amino acids, a bicarbonate, a carbonate, a hydroxide, or a combination thereof. For example, the crosslinking agent(s) may be selected from polyphosphates, basic amino acids, a bicarbonate, a carbonate, or a combination thereof. In some instances, the crosslinking agent(s) comprise a polyphosphate, a basic amino acid, or a combination thereof. Preferably, the crosslinking agent comprises a polyphosphate.

In certain preferred embodiments, the oral care composition comprises a phosphate/acrylate copolymer and, optionally, a carbomer. The phosphate/acrylate copolymer may be a carboxylic acid polymer having at least one phosphate moiety, wherein the monomeric unit comprises acrylic acid, methacrylic acid, 2-hydroxyethyl methacrylate, or any combinations thereof having at least one phosphate moiety. For example, the phosphate/acrylate copolymer may comprise or be formed of: from about 5 to about 30 mol. %, optionally about 8 to about 14 mol %, of 2-hydroxyethyl methacrylate phosphate monomer; from about 60 to about 90 mol. %, optionally about 65 to about 85 mol. %, optionally about 71 to about 79 mol., of acrylic acid monomer; from about 5 to about 35 mol. %, optionally about 10 to about 25 mol. %, optionally about 10 to about 19 mol. %, of methacrylic acid monomer. Non-limiting examples of phosphate/acrylate copolymers include those according to Table 1.

TABLE 1

| | Monomer Weight | Monomer Molar |
|---|---|---|
| Monomer Name and Structure | Ratio (weight %) | Ratio (Mole %) |
| 2-hydroxyethyl methacrylate phosphate | 11 | 4 |
| 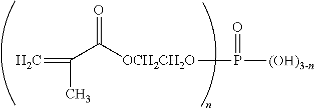 | | |
| mixture of n = 0, n = 1, and n = 2 | | |
| acrylic acid | 75 | 85 |
| 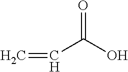 | | |
| methacrylic acid | 14 | 11 |
| 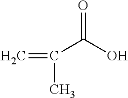 | | |

In some embodiments, the phosphate/acrylate copolymer is a copolymerized product of a mixture of acrylic acid, methacrylic acid, and 2-hydroxyethyl methacrylate phosphates, with the copolymerized product having acrylic acid in a molar percentage of about 70 to about 90 mol. %, about 80 to about 90 mol. %, or about 85 mol. %; methacrylic acid in a molar percentage of about 5 to about 20 mol. %, about 5 to about 15 mol. %, or about 11 mol %; and hydroxyethyl methacrylate phosphates in a molar percentage of about 1 about 10 mol. %, about 2 about 6 mol. %, or about 4 mol. %. In at least one embodiment, the phosphate/acrylate copolymer comprises or consists of DV8801 polymer (which may sometimes be referred to as Mirapol 8801).

In some embodiments, the phosphate/acrylate copolymer has a weight average molecular weight of from about 10 to about 500 kDa, such as about 10 to about 200 kDa, about 10 to about 40 kDa, about 15 to about 25 kDa, or about 17 to about 23 kDa. In certain embodiments, the weight average molecular weight is about 10 to about 40 kDa. In other embodiments, the weight average molecular weight is about 17 to about 23 kDa. For example, in some embodiments, the phosphate/acrylate copolymer is a random copolymer that is the copolymerized product of a mixture of, e.g., acrylic acid, methacrylic acid, and 2-hydroxyethyl methacrylate in the relative amounts discussed above.

The total amount of phosphate/acrylate copolymer in the oral care composition may in some cases be from about 0.1 to about 10 wt. %, based on the total weight of the oral care composition. For example, the total amount of phosphate/acrylate copolymer present in the oral care composition may be from about 0.1 to about 7 wt. %, about 0.1 to about 5 wt. %, about 0.1 to about 3 wt. %, about 0.1 to about 1 wt. %; from about 0.5 to about 10 wt. %, about 0.5 to about 7 wt. %, about 0.5 to about 5 wt. %, about 0.5 to about 3 wt. %, about 0.5 to about 1.5 wt. %; from about 0.1 to about 10 wt. %, about 0.1 to about 7 wt. %, about 0.1 to about 5 wt. %, about 0.1 to about 3 wt. %, about 0.1 to about 1 wt. %; from about 1 to about 10 wt. %, about 1 to about 7 wt. %, about 1 to about 5 wt. %, about 1 to about 3 wt. %, about 1 to about 2 wt. %; from about 2 to about 10 wt. %, about 2 to about 7 wt. %, about 2 to about 5 wt. %; from about 4 to about 10 wt. %, about 4 to about 7 wt. %; from about 6 to about 10 wt. %, about 6 to about 8 wt. %, or any range or subrange thereof, based on the total weight of the oral care composition.

The oral care composition may comprise a polyphosphate an effective amount. In some embodiments, the amount of polyphosphate present in the oral care composition is from about 0.1 to about 7 wt. %, about 0.1 to about 6 wt. %, about 0.1 to about 5 wt. %, about 0.1 to about 4 wt. %, about 0.1 to about 3 wt. %, about 0.1 to about 2.7 wt. %, about 0.1 to about 2.4 wt. %, about 0.1 to about 2.1 wt. %, about 0.1 to about 1.7 wt. %, about 0.1 to about 1.4 wt. %, about 0.1 to about 1.1 wt. %; from about 0.5 to about 7 wt. %, about 0.5 to about 5 wt. %, about 0.5 to about 3 wt. %, about 0.5 to about 2.7 wt. %, about 0.5 to about 2.4 wt. %, about 0.5 to about 2.1 wt. %, about 0.5 to about 1.7 wt. %, about 0.5 to about 1.4 wt. %, about 0.5 to about 1.1 wt. %; from about 1 to about 7 wt. %, about 1 to about 5 wt. %, about 1 to about 4 wt. %, about 1 to about 3 wt. %, about 1 to about 2.7 wt. %, about 1 to about 2.4 wt. %, about 1 to about 2.1 wt. %, about 1 to about 1.7 wt. %; from about 1.3 to about 7 wt. %, about 1.3 to about 5 wt. %, about 1.3 to about 4 wt. %, about 1.3 to about 3 wt. %, about 1.3 to about 2.7 wt. %, about 1.3 to about 2.4 wt. %, about 1.3 to about 2.1 wt. %, about 1.3 to about 1.7 wt. %; from about 1.6 to about 7 wt. %, about 1.6 to about 5 wt. %, about 1.6 to about 4 wt. %, about 1.6 to about 3 wt. %, about 1.6 to about 2.7 wt. %, about 1.6 to about 2.4 wt. %, about 1.6 to about 2.1 wt. %; from about 1.9 to about 7 wt. %, about 1.9 to about 5 wt. %, about 1.9 to about 4 wt. %, about 1.9 to about 3 wt. %, about 1.9 to about 2.7 wt. %, about 1.9 to about 2.4 wt. %; from about 2.4 to about 7 wt. %, about 2.4 to about 6 wt. %, about 2.4 to about 5 wt. %, about 2.4 to about 4 wt. %, about 2.4 to about 3 wt. %, about 2.4 to about 2.7 wt. %; from about 2.7 to about 7 wt. %, about 2.7 to about 6 wt. %, about 2.7 to about 5 wt. %, about 2.7 to about 4 wt. %; from about 3.3 to about 7 wt. %, about 3.3 to about 6 wt. %, about 3.3 to about 5 wt. %, about 3.3 to about 4 wt. %, from about 3.9 to about 7 wt. %, about 3.9 to about 6 wt. %, about 3.9 to about 5 wt. %; from about 4.6 to about 7 wt. %, about 4.6 to about 6 wt. %, about 4.6 to about 5 wt. %; from about 5.5 to about 7 wt. %, about 5.5 to about 6.5 wt. %, or any range or subrange thereof, based on the total weight of the oral care composition.

The polyphosphate may comprise, or in some cases consist of, polyphosphates selected from pyrophosphates, orthophosphates, polymetaphosphates, and a combination of two or more thereof. The polyphosphate may comprise a pyrophosphate, such as an alkali metal pyrophosphate selected from a tetra alkali metal pyrophosphate, dialkali metal diacid pyrophosphate, trialkali metal monoacid pyrophosphate and mixtures thereof. The alkali metals may be sodium and/or potassium. For example, the polyphosphate may be an alkali metal polyphosphate selected from a pyrophosphate, a tripolyphosphate, a tetraphosphate, a hexametaphosphate, and a combination thereof. In some embodiments, the polyphosphate is selected from sodium pyrophosphate, potassium pyrophosphate, sodium tripolyphosphate, potassium tripolyphosphate, or a combination thereof. In further embodiments, the polyphosphate comprises tetrasodium pyrophosphate, tetrapotassium pyrophosphate, sodium tripolyphosphate, tetrapolyphosphate, sodium trimetaphosphate, sodium hexametaphosphate, potassium polyphosphate, sodium polyphosphate, or a combination of two or more thereof.

In certain embodiments, the oral care composition is preferably formulated to have a weight ratio of polyacrylic acid and/or a salt thereof to polyphosphate of about 1:3 to about 1:8. For example, the weight ratio of polyacrylic acid and/or a salt thereof to polyphosphate may be about 1:3 to about 1:8, about 1:3 to about 1:7, about 1:3 to about 1:6, about 1:3 to about 1:5, about 1:3 to about 1:4; from about 1:4 to about 1:8, about 1:4 to about 1:7, about 1:4 to about 1:6, about 1:4 to about 1:5; from about 1:5 to about 1:8, about 1:5 to about 1:7, about 1:5 to about 1:6; from about 1:6 to about 1:8, about 1:6 to about 1:7, or about 1:7 to about 1:8, including any range or subrange thereof.

The oral care composition may comprise one or more basic amino acid, preferably in an effective amount. In some embodiments, the one or more amino acid(s) may be present in the oral care composition in an amount from about 0.1 to about 10 wt. %, about 0.1 to about 8 wt. %, about 0.1 to about 6 wt. %, about 0.1 to about 4 wt. %, about 0.1 to about 2 wt. %, about 0.1 to about 1 wt. %, about 0.1 to about 0.5 wt. %; from about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 2 wt. %; from about 2 to about 10 wt. %, about 2 to about 8 wt. %, about 2 to about 6 wt. %, about 2 to about 4 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %; from about 6 to about 10 wt. %, about 6 to about 8 wt. %; from about 8 to about 10 wt. %, including any range or subrange thereof, based on the total weight of the oral care composition.

The one or more basic amino acids may be selected from naturally occurring basic amino acids, such as arginine, lysine, and histidine, and non-naturally occurring basic amino acids having a carboxyl group and an amino group in the molecule, which are water-soluble and provide an aqueous solution with a pH of 7 or greater. The one or more basic amino acid may be selected from lysine, arginine, citrulline, ornithine, and a combination of two or more thereof. For instance, the personal care composition may comprise one or more of L-lysine, L-arginine, D-lysine, D-glycine, or a combination of two or more thereof. In at least one embodiment, the basic amino acid is selected from L-glycine, L-arginine, and a combination thereof. In some embodiments, the only amino acids in the oral care composition are basic amino acids, such as those disclosed herein.

Examples of additional amino acids that may be incorporated or excluded from the oral care composition include glycine, serine, citrulline, ornithine, creatine, histidine, diaminobutanoic acid, diaminoproprionic acid, salts thereof or combinations thereof. Additionally or alternatively, the oral care composition may include a neutral amino acids, such as those selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

The oral care composition may comprise a bicarbonate, a carbonate, or a combination thereof. The bicarbonate, carbonate, or combination thereof may be in the form of a salt having a cation selected from potassium, sodium, and calcium. Examples of bicarbonates, carbonates, or combinations thereof that may be incorporated into, or in some cases excluded from, the oral care composition includes sodium bicarbonate, potassium bicarbonate potassium carbonate, calcium carbonate, or a combination thereof.

The total amount of bicarbonate and/or carbonate in the oral care composition may be from about 0.1 to about 4 wt. %, about 0.1 to about 2.5 wt. %, about 0.1 to about 2 wt. %, about 0.1 to about 1.5 wt. %, about 0.1 to about 1.25 wt. %, about 0.1 to about 1 wt. %, about 0.1 to about 0.75 wt. %, about 0.1 to about 0.5 wt. %, about 0.1 to about 0.4 wt. %, about 0.1 to about 0.3 wt. %; from about 0.2 to about 4 wt. %, about 0.2 to about 2.5 wt. %, about 0.2 to about 2 wt. %, about 0.2 to about 1.5 wt. %, about 0.2 to about 1.25 wt. %, about 0.2 to about 1 wt. %, about 0.2 to about 0.75 wt. %, about 0.2 to about 0.5 wt. %; from about 0.35 to about 4 wt. %, about 0.35 to about 2.5 wt. %, about 0.35 to about 2 wt. %, about 0.35 to about 1.5 wt. %, about 0.35 to about 1.25 wt. %, about 0.35 to about 1 wt. %, about 0.35 to about 0.75 wt. %, about 0.35 to about 0.5 wt. %; from about 0.5 to about 4 wt. %, about 0.5 to about 2.5 wt. %, about 0.5 to about 2 wt. %, about 0.5 to about 1.5 wt. %, about 0.5 to about 1.25 wt. %, about 0.5 to about 1 wt. %, about 0.5 to about 0.75 wt. %; from about 1 to about 4 wt. %, about 1 to about 3 wt. %, about 1 to about 2.5 wt. %, about 1 to about 2 wt. %, about 1 to about 1.5 wt. %, about 1 to about 1.25 wt. %; from about 1.5 to about 4 wt. %, about 1.5 to about 3 wt. %, about 1.5 to about 2.5 wt. %, about 1.5 to about 2 wt. %; from about 2 to about 4 wt. %, about 2 to about 3 wt. %, about 2 to about 2.5 wt. %; from about 2.5 to about 4 wt. %, about 2.5 to about 3 wt. %, about 3 to about 4 wt. %, or any range or subrange thereof, based on the total weight of the oral care composition.

The oral care composition may comprise a hydroxide. Examples of hydroxides that may be included in, or in some cases excluded from, the oral care composition include sodium hydroxide, calcium hydroxide, magnesium hydroxide, and potassium hydroxide. The total amount of hydroxide in the oral care composition may be from about 0.1 to about 4 wt. %, about 0.1 to about 2.5 wt. %, about 0.1 to about 2 wt. %, about 0.1 to about 1.5 wt. %, about 0.1 to about 1.25 wt. %, about 0.1 to about 1 wt. %, about 0.1 to about 0.75 wt. %, about 0.1 to about 0.5 wt. %, about 0.1 to about 0.4 wt. %, about 0.1 to about 0.3 wt. %; from about 0.2 to about 4 wt. %, about 0.2 to about 2.5 wt. %, about 0.2 to about 2 wt. %, about 0.2 to about 1.5 wt. %, about 0.2 to about 1.25 wt. %, about 0.2 to about 1 wt. %, about 0.2 to about 0.75 wt. %, about 0.2 to about 0.5 wt. %; from about 0.35 to about 4 wt. %, about 0.35 to about 2.5 wt. %, about 0.35 to about 2 wt. %, about 0.35 to about 1.5 wt. %, about 0.35 to about 1.25 wt. %, about 0.35 to about 1 wt. %, about 0.35 to about 0.75 wt. %, about 0.35 to about 0.5 wt. %; from about 0.5 to about 4 wt. %, about 0.5 to about 2.5 wt. %, about 0.5 to about 2 wt. %, about 0.5 to about 1.5 wt. %, about 0.5 to about 1.25 wt. %, about 0.5 to about 1 wt. %, about 0.5 to about 0.75 wt. %; from about 1 to about 4 wt. %, about 1 to about 3 wt. %, about 1 to about 2.5 wt. %, about 1 to about 2 wt. %, about 1 to about 1.5 wt. %, about 1 to about 1.25 wt. %; from about 1.5 to about 4 wt. %, about 1.5 to about 3 wt. %, about 1.5 to about 2.5 wt. %, about 1.5 to about 2 wt. %; from about 2 to about 4 wt. %, about 2 to about 3 wt. %, about 2 to about 2.5 wt. %; from about 2.5 to about 4 wt. %, about 2.5 to about 3 wt. %, about 3 to about 4 wt. %, or any range or subrange thereof, based on the total weight of the oral care composition.

The oral care compositions typically comprise an amount of water. The amount of water present in the oral care compositions may be about 10 wt. % or more, about 15 wt. % or more, about 20 wt. % or more, about 25 wt. % or more, about 30 wt. % or more, about 35 wt. % or more or any range or subrange thereof (e.g., about 10 to 35 wt. %), based on the total weight of the oral care composition. In some embodiments, the oral care composition comprises water in an amount of e.g., preferably, from about 10 to about 45 wt. %, about 10 to about 40 wt. %, about 10 to about 35 wt. %, about 10 to about 32 wt. %, about 10 to about 29 wt. %, about 10 to about 26 wt. %, about 10 to about 23 wt. %, about 10 to about 17 wt. %; from about 15 to about 45 wt. %, about 15 to about 40 wt. %, about 15 to about 35 wt. %, about 15 to about 32 wt. %, about 15 to about 29 wt. %, about 15 to about 26 wt. %, about 15 to about 23 wt. %, about 15 to about 19 wt. %; from about 19 to about 45 wt. %, about 19 to about 40 wt. %, about 19 to about 35 wt. %, about 19 to about 32 wt. %, about 19 to about 29 wt. %, about 19 to about 26 wt. %, about 19 to about 23 wt. %; from about 22 to about 45 wt. %, about 22 to about 40 wt. %, about 22 to about 35 wt. %, about 22 to about 32 wt. %, about 22 to about 29 wt. %, about 22 to about 26 wt. %; from about 25 to about 45 wt. %, about 25 to about 40 wt. %, about 25 to about 35 wt. %, about 25 to about 32 wt. %, about 25 to about 29 wt. %; from about 28 to about 45 wt. %, about 28 to about 40 wt. %, about 28 to about 35 wt. %, about 28 to about 32 wt. %; from about 31 to about 45 wt. %, about 31 to about 40 wt. %, about 31 to about 35 wt. %; from about 34 to about 45 wt. %, about 34 to about 40 wt. %, about 37 to about 45 wt. %, or any range or subrange thereof, based on the total weight of the oral care composition.

A potassium source may be included in the oral care compositions, preferably in an effective amount. The amount of potassium source may be from about 0.1 to about 6 wt. %, based on the total weight of the oral care composition. For instance, the oral care composition may include a potassium source in an amount from about 0.1 to about 6 wt. %, about 0.1 to about 4 wt. %, about 0.1 to about 3 wt. %, about 0.1 to about 2 wt. %, about 0.1 to about 1.5 wt. %, about 0.1 to about 1 wt. %, about 0.1 to about 0.5 wt. %; from about 0.3 to about 5 wt. %, about 0.3 to about 4 wt. %, about 0.3 to about 3 wt. %, about 0.3 to about 2 wt. %, about 0.3 to about 1.5 wt. %, about 0.3 to about 1 wt. %; from about 0.5 to about 5 wt. %, about 0.5 to about 4 wt. %, about 0.5 to about 3 wt. %, about 0.5 to about 2 wt. %, about 0.5 to about 1.5 wt. %, about 0.5 to about 1 wt. %; from about 0.9 to about 5 wt. %, about 0.9 to about 4 wt. %, about 0.9 to about 3 wt. %, about 0.9 to about 2 wt. %, about 0.9 to about 1.5 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 6 wt. %, about 2 to about 5 wt. %, about 2 to about 4 wt. %, about 2 to about 3 wt. %; from about 3 to about 6 wt. %, about 3 to about 5 wt. %; from about 4 to about 6 wt. %, about 4 to about 5 wt. %; from about 5 to about 6 wt. %, or any range or subrange thereof, based on the total weight of the oral care composition.

The potassium source may be a potassium ion source. The potassium source typically comprises potassium nitrate, potassium citrate, or a combination thereof. Preferably, the oral care composition comprises potassium nitrate. In at least one embodiment, the potassium ion source consists essentially of or consists of potassium nitrate.

Additionally or alternatively, the oral care composition may comprise one or more nitrate source (e.g., nitrate ion source), preferably present in an effective amount. The nitrate source(s) may comprise or consist of nitrate ion source(s). The nitrate source(s) (e.g., nitrate ion source(s)) may be present in the oral care composition in an amount from about 0.1 to about 8 wt. %, based on the total weight of the oral care composition. In some instances, the amount of nitrate source (e.g., nitrate ion source) present in the oral care composition may be from about 0.1 to about 6 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %; from about 5 to about 8 wt. %, about 5 to about 6 wt. %, or any range or subrange thereof, based on the total weight of the oral care composition.

The one or more nitrate source (e.g., nitrate ion source) may be in the form of a salt or an ion derived therefrom, including, e.g., nitrate salt is selected from an alkali or alkaline earth metal nitrate. Examples of nitrate sources (e.g., nitrate ion sources) include lithium nitrate, sodium nitrate, potassium nitrate, magnesium nitrate, calcium nitrate, zinc nitrate, silver nitrate, ammonium nitrate, or a combination two or more thereof. In at least one preferred embodiment, the nitrate source comprises potassium nitrate.

The oral care compositions may be formulated to have a molar ratio of nitrate ions to stannous ions, both measured as free ions, that is about 2:1 or less. For example, the oral care composition may have a molar ratio of nitrate ions to stannous ions, both measured as free ions, of from about 0.5:1 to about 2:1, about 0.5:1 to about 1.8:1, about 0.5:1 to about 1.6:1, about 0.5:1 to about 1.4:1, about 0.5:1 to about 1.2:1, about 0.5:1 to about 1:1; from about 0.7:1 to about 2:1, about 0.7:1 to about 1.8:1, about 0.7:1 to about 1.6:1, about 0.7:1 to about 1.4:1, about 0.7:1 to about 1.2:1, about 0.7:1 to about 1:1; from about 0.9:1 to about 2:1, about 0.9:1 to about 1.8:1, about 0.9:1 to about 1.6:1, about 0.9:1 to about 1.4:1, about 0.9:1 to about 1.2:1, about 0.9:1 to about 1:1, or any range or subrange thereof. In some embodiments, the oral care composition is formulated to have a molar ratio of nitrate ions to stannous ions, both measured as free ions, of about 1:1.

The oral care compositions may include one or more polyol(s). The one or more polyol(s) may be present in the oral care composition in an amount from about 10 to about 58 wt. %, based on the total weight of the oral care composition. For example, the total amount of polyol(s) present in the oral care composition may be from about 10 to about 55 wt. %, about 10 to about 45 wt. %, about 10 to about 37 wt. %, about 10 to about 31 wt. %, about 10 to about 25 wt. %; from about 15 to about 60 wt. %, about 15 to about 55 wt. %, about 15 to about 45 wt. %, about 15 to about 37 wt. %, about 15 to about 31 wt. %, about 5 to about 25 wt. %; from about 20 to about 58 wt. %, about 20 to about 54 wt. %, about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 58 wt. %, about 25 to about 54 wt. %, about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 58 wt. %, about 28 to about 54 wt. %, about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 58 wt. %, about 31 to about 54 wt. %, about 31 to about 50 wt. %, about 31 to about 45 wt. %, about 31 to about 40 wt. %, about 31 to about 37 wt. %; from about 34 to about 58 wt. %, about 34 to about 54 wt. %, about 34 to about 50 wt. %, about 34 to about 45 wt. %, about 34 to about 40 wt. %; from about 37 to about 58 wt. %, about 37 to about 54 wt. %, about 37 to about 50 wt. %, about 37 to about 45 wt. %; from about 40 to about 58 wt. %, about 40 to about 54 wt. %, about 40 to about 50 wt. %, about 40 to about 45 wt. %; from about 43 to about 58 wt. %, about 43 to about 54 wt. %, about 43 to about 50 wt. %, about 43 to about 48 wt. %; from about 45 to about 58 wt. %, about 45 to about 54 wt. %, about 45 to about 50 wt. %, about 45 to about 48 wt. %, from about 48 to about 58 wt. %, about 48 to about 54 wt. %, about 48 to about 50 wt. %, from about 52 to about 58 wt. %, about 52 to about 56 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). In some preferred embodiments, the polyol(s) comprises glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, or a combination or two or more thereof. The polyol may be a humectant including, e.g., glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer, and a combination of two or more thereof.

In some cases, the oral care composition includes one or more polyol selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, and dipropylene glycol, or a combination of two or more thereof. Additional, non-limiting examples of polyols that may, optionally, be included in the oral care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture of two or more thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600. In some embodiments, the oral care composition comprises one or more polyethylene glycols, e.g., polyethylene glycols in a molecular weight range from 200 to 800. For example, the compositions may comprise one or more of polyethylene glycol 200, polyethylene glycol 300, polyethylene glycol 400, polyethylene glycol, 600 or polyethylene glycol 800.

In certain embodiments, the oral care composition comprises two or more polyols. In some instances, at least one of the polyols is selected glycerin and sorbitol. In at least one embodiment, the oral care composition comprises glycerin and sorbitol.

An organic acid buffer system may be included in certain embodiments of the oral care composition. The organic acid buffer system may comprise citric acid and/or a salt thereof. For instance, the oral care composition may include a salt of citric acid, such as sodium citrate. Examples of sodium citrate include trisodium citrate, disodium citrate, and mono citrate. In certain embodiments, the oral care composition includes citric acid and a salt thereof (e.g., sodium citrate).

The oral care composition may be formulated to have an organic acid buffer system comprising a weight ratio of citric acid to sodium citrate (such as, trisodium citrate, disodium citrate, mono citrate, or a combination thereof) from about 1:3 to about 1:8. For instance, the weight ratio of citric acid to sodium citrate may be about 1:3 to about 1:8, about 1:3 to about 1:7, about 1:3 to about 1:6, about 1:3 to about 1:5, about 1:3 to about 1:4; from about 1:4 to about 1:8, about 1:4 to about 1:7, about 1:4 to about 1:6, about 1:4 to about 1:5; from about 1:5 to about 1:8, about 1:5 to about 1:7, about 1:5 to about 1:6; from about 1:6 to about 1:8, about 1:6 to about 1:7, or about 1:7 to about 1:8, including any range or subrange thereof.

In some embodiments, the amount of sodium citrate in the oral care composition is from about 0.1 to about 8 wt. %, about 0.1 to about 6 wt. %, about 0.1 to about 4 wt. %, about 0.1 to about 2 wt. %, about 0.1 to about 1 wt. %, about 0.1 to about 0.5 wt. %; from about 0.5 to about 8 wt. %, about 0.5 to about 6 wt. %, about 0.5 to about 4 wt. %, about 0.5 to about 2 wt. %, about 0.5 to about 1 wt. %; from about 1 to about 8 wt. %, about 1 to about 6 wt. %, about 1 to about 4 wt. %, about 1 to about 2 wt. %; from about 2 to about 8 wt. %, about 2 to about 6 wt. %, about 2 to about 4 wt. %; from about 3 to about 8 wt. %, about 3 to about 6 wt. %, about 3 to about 4 wt. %; from about 4 to about 8 wt. %, about 4 to about 6 wt. %, about 6 to about 8 wt. %, including any range or subrange thereof, based on the total weight of the oral care composition.

Additionally or alternatively, the amount of citric acid in the oral care composition may be from about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %, about 3 to about 5 wt. %, or any range or subrange thereof, based on the total weight of the oral care composition.

The oral care composition may include one or more thickening agent(s) other than the polyacrylic acid and/or a salt thereof. The amount of thickening agent(s) other than a polyacrylic acid, a phosphate/acrylate copolymer, and/or salt(s) thereof may be from about 0.1 to about 8 wt. %, based on the total weight of the oral care composition. For example, the amount of thickening agent(s) other than polyacrylic acid, a phosphate/acrylate copolymer, and/or salt(s) thereof in the oral care composition may be from about 0.1 to about 6 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %; from about 5 to about 8 wt. %, about 5 to about 6 wt. %, or any range or subrange thereof, based on the total weight of the oral care composition.

As mentioned above, in certain embodiments, the oral care composition may have a reduced amount of thickening agents other than those selected from a polyacrylic acid, a phosphate/acrylate copolymer, a salt thereof, or combinations thereof. For instance, the oral care composition may comprise thickening agents other than a polyacrylic acid, or a phosphate/acrylate copolymer, and/or salt(s) thereof in an amount from about 0.1 to about 2.5 wt. % about 0.3 to about 2.5 wt. %, about 0.6 to about 2 wt. %, about 1 to about 2.5 wt. %; from about 0.1 to about 2 wt. % about 0.3 to about 2 wt. %, about 0.6 to about 2 wt. %, about 1 to about 2 wt. %; from about 0.1 to about 1.5 wt. % about 0.3 to about 1.5 wt. %, about 0.6 to about 1.5 wt. %, or any range or subrange thereof. from about 0.1 to about 1 wt. % about 0.3 to about 1 wt. %, about 0.6 to about 1 wt. %, or any range or subrange thereof, based on the total weight of the oral care composition.

Thickening agents may be referred to as “thickeners” or “viscosity modifying agents.” Thickening agents are typically included to increase the viscosity of the oral care compositions. Nonetheless, in some instances, certain thickening agents provide additional, surprising benefits to the oral care compositions.

The one or more thickening agent(s) other than polyacrylic acid, a phosphate/acrylate copolymer, and/or salt(s) thereof, which may be included or excluded from embodiments of the oral care composition, include those selected from a polysaccharide, a silica thickener, an acrylate, a polymer other than other than polyacrylic acid, a phosphate/acrylate copolymer, and/or salt(s) thereof, and a combination of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, carrageenan, or a combination of two more thereof. Natural gums such as karaya, gum arabic, xanthan gum, and gum tragacanth may also be incorporated or excluded from certain embodiments of the oral care composition. Colloidal magnesium aluminum silicate can also be used as a component of the thickening composition to further improve the composition's texture. Silica thickeners, which form polymeric structures or gels in aqueous media, may be present. Silica thickeners are typically physically and functionally distinct from the particulate silica abrasives as the silica thickeners are very finely divided and provide little or no abrasive action.

In some embodiments, the one or more thickening agent(s) comprises a polymer selected from polysaccharides, acrylates, polyvinyl pyrrolidone, polyitaconates, an acrylamides, and combinations thereof. Polyvinyl pyrrolidone generally refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The polyvinyl pyrrolidone may have an average molecular weight in the range 5,000 to 100,000, preferably in the range 5,000 to 50,000. Polyvinyl pyrrolidones that have average molecular weights of 10,000, 30,000 and 40,000 may be commercially available from Sigma Chemical Co., GAF Corporation and Sigma Chemical Co. In some embodiments, the polymer comprises crosslinked polyvinyl pyrrolidone (crosslinked PVP). In at least one embodiment, the polymer consists of polyvinyl pyrrolidone.

Examples of acrylates that may be included in the oral care composition include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include trimethylol propane, ethoxylated, glyceryl propoxy, and pentaerythritol.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate, trifluoroethyl methacrylate, or a combination of two or more thereof.

Examples of acrylamides include, but are not limited to, acrylamide, methacrylamide and di(C₁-C₃₀) alkyl-acrylamides and -methacrylamides such as those of methyl, ethyl, propyl, butyl, pentyl, hexyl and the like. N-substituted acrylamides that may be suitable include N-ethylacrylamide, N-tert-butylacrylamide, N-tert-octylacrylamide, N-octylacrylamide, N-decylacrylamide, N-dodecylacrylamide and the corresponding N-substituted methacrylamides. Other N-substituted acrylamides include N-hydroxymethyl acrylamide, N-isopropylacrylamide, N-methylacrylamide, N,N′-methylenebisacrylamide, N-isobutoxymethylacrylamide, N,N-dimethylacrylamide, and 2-acrylamido-2-methylpropanesulfonic acid. In some embodiments, one or more of the above listed thickening agents other than polyacrylic acid and/or a salt thereof are excluded from the oral care composition.

The oral care compositions may include a fluoride source (e.g., a fluoride ion source). The fluoride source may comprise or consist of fluoride ion source(s). The fluoride source may be present in an effective amount. In some cases, the fluoride source (e.g., fluoride ion source) is present in the oral care composition in an amount of about 0.01 to about 5 wt. %, based on the total weight of the oral care composition. For example, the fluoride source (e.g., fluoride ion source) is present in an amount of about 0.01 to about 5 wt. %, about 0.01 to about 4 wt. %, about 0.01 to about 3 wt. %, about 0.01 to about 2 wt. %, about 0.01 to about 1 wt. %; from about 0.05 to about 5 wt. %, about 0.05 to about 4 wt. %, about 0.05 to about 3 wt. %, about 0.05 to about 2 wt. %, about 0.05 to about 1 wt. %; from about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The fluoride source (e.g., fluoride ion source) may be selected from soluble fluoride ion salts. For example, the fluoride source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride source (e.g., fluoride ion source) comprises sodium fluoride, sodium monofluorophosphate, or a combination thereof. Additional examples of fluoride ion source are disclosed in U.S. Pat. Nos. 3,535,421, 4,885,155, and 3,678,154, the disclosure of each of which is hereby incorporated by reference in their entirety.

In certain embodiments, the oral care composition of the disclosure may contain stannous fluoride and a source of fluoride ions or fluorine-providing agents in amounts sufficient to deliver, in total, from 25 ppm to 25,000 ppm (mass fraction) of fluoride ions, generally at least 500 ppm, e.g., from about 500 to about 2000 ppm, from about 800 to about 1800 ppm, from about 1000 to about 1600 ppm, from about 1200 to about 1550 ppm, or about 1450 ppm.

The oral care compositions may include one or more abrasive(s), e.g., in an amount from about 5 to about 35 wt. %, based on the total weight of the oral care composition. In some embodiments, the one or more abrasive(s) is present in an amount from about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the oral care composition.

The one or more abrasive(s) may include: silica, silicate, silicon, alumina (including calcined aluminum oxide), aluminosilicates, such as bentonite, zeolite, kaolin, and mica, siliceous or diatomaceous earth, pumice, calcium carbonate, cuttlebone, insoluble phosphates, composite resins, such as melamine resin, phenolic resin, and urea-formaldehyde resin, polycarbonate, silicon carbide, boron carbide, microcrystalline wax, microcrystalline cellulose, including combinations of colloidal microcrystalline cellulose and carboxymethylcellulose, and combinations and derivatives thereof. For example, the one or more abrasive may be selected from silicas including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, resinous abrasive materials such as particulate condensation products of urea and formaldehyde, and combinations thereof.

As used herein, “mica” refers to any of a group of hydrous aluminum silicate minerals with plate morphology and/or perfect basal (micaceous) cleavage. Mica can be, for example, sheet mica, scrap mica or flake mica, as exemplified by muscovite, biotite or phlogopite type micas. The abrasive may be selected from insoluble phosphates, such as orthophosphates, polymetaphosphates, pyrophosphates, and a combination thereof. Synthetic silicas include both silica gels and precipitated silicas that are prepared by the neutralization of aqueous silicate solutions with a strong mineral acid. Abrasives comprising silica may be useful in certain embodiments of the oral care composition. In certain embodiments, the oral compositions may comprise a particularly efficacious combination of silica abrasive particle species. For example, the abrasive(s) be selected from high cleaning silica.

The oral care compositions may include an abrasive system comprising two or more abrasives. For example, an abrasive system comprise 2 to 7, 2 to 6, 2 to 5, 2 to 5, or 2 to 4; 3 to 7, 3 to 6, 2 to 5, or 3 to 5 abrasives. The abrasives may comprise one or more cleaning abrasive and/or one or more polishing abrasives. As appreciated by one of skill in the art, a single abrasive species typically performs at least some cleaning and polishing simultaneously. However, particles are generally categorized in the art by the predominant effect they have on a target oral surface. Typically, “polishing abrasives” are considered to be relatively small particles having high hardness, where abrasives with relatively large particle sizes and low hardness are considered to be “cleaning abrasives.” In certain embodiments, the oral care composition comprises two or more abrasives comprising silica. In some embodiments, the first abrasive is selected to be a harder and smaller abrasive, e.g., a higher cleaning and/or polishing abrasive, and the second abrasive is a typical cleaning abrasive. In some embodiments, the oral care composition includes at least one polishing abrasive and/or at least one cleaning abrasive. Further examples of abrasives are disclosed in U.S. Patent Publication No. 2007/140986, which is incorporated herein in its entirety by reference for all purposes.

Surfactants may be included in the oral care composition. The one or more surfactant(s) may be present in an amount ranging from about 0.5 to about 8 wt. %, based on the total weight of the oral care composition. For example, the oral care composition may include one or more surfactant in an amount from about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 9 wt. %, about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 2 to about 9 wt. %, about 2 to about 8 wt. %, about 2 to about 7 wt. %, about 2 to about 6 wt. %, about 2 to about 5 wt. %, about 2 to about 4 wt. %, about 2 to about 3 wt. %; from about 2.5 to about 9 wt. %, about 2.5 to about 8 wt. %, about 2.5 to about 7 wt. %, about 2.5 to about 6 wt. %, about 2.5 to about 5 wt. %, about 2.5 to about 4 wt. %, about 2.5 to about 3.5 wt. %; from about 3 to about 9 wt. %, about 3 to about 8 wt. %, about 3 to about 7 wt. %, about 3 to about 6 wt. %, about 3 to about 5 wt. %, about 3 to about 4 wt. %, about 3 to about 3.5 wt. %; from about 4 to about 9 wt. %, about 4 to about 8 wt. %, about 4 to about 7 wt. %, about 4 to about 6 wt. %, about 4 to about 5 wt. %; from about 5 to about 9 wt. %, about 5 to about 8 wt. %, about 5 to about 7 wt. %, about 5 to about 6 wt. %; from about 7 to about 9 wt. %, about 7 to about 8 wt. %, or any range or subrange thereof, based on the total weight of the oral care composition.

The one or more surfactants may be selected from anionic surfactants, such as sulfate based anionic surfactants, sulfonated monoglycerides of fatty acids, acyl isethionates, acyl sarcosinates, acyl taurate, or combinations thereof. Examples of sulfate based anionic surfactants include ammonium lauryl sulfate, ammonium lauryl ether sulfate, sodium dodecyl sulfate, sodium coco-sulfate, ammonium coco-sulfate, or a combination of two or more thereof. The one or more anionic surfactant(s) may be selected from non-sulfate based anionic surfactants, such as sulfonated monoglycerides of fatty acids, acyl isethionates, acyl sarcosinates, acyl taurate, or a combination of two or more thereof. For example, the anionic surfactants may be selected from water-soluble salts of higher fatty acid monoglyceride monosulfates, such as the sodium salt of the monosulfated monoglyceride of hydrogenated coconut oil fatty acids; sodium N-methyl N-cocoyl taurate; sodium cocomo-glyceride sulfate; higher alkyl sulfates, such as sodium lauryl sulfate; higher alkyl-ether sulfates, such as sodium laureth-2 sulfate; higher alkyl aryl sulfonates, such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfolaurate (N-2-ethyl laurate potassium sulfacetamide) and sodium lauryl sarcosinate.

The one or more surfactants may be chosen from non-ionic, amphoteric, and cationic surfactants. Nonionic surfactants useful herein may include those compounds produced by the condensation of alkylene oxide groups (hydrophilic in nature) with an organic hydrophobic compound, which may be aliphatic or alkyl-aromatic in nature. Non-limiting examples of suitable nonionic surfactants include polyoxyethylene sorbitan esters (sold under the trade name Tweens), polyoxyl 40 hydrogenated castor oil, fatty alcohol ethoxylates, ethylene oxide condensates of aliphatic alcohols, long chain tertiary amine oxides, long chain tertiary phosphaine oxides, lauryl glucoside (sold under the trade name Plantaren 1200 UP) and long chain dialkyl sulfoxides. Suitable nonionic surfactants with a HLB of 7 or more include sucrose laurate, sucrose cocoate, sucrose stearate; Steareth 20, 21, or 100, and PEG 20 Sorbitan Monostearate (commercially available as Tween 60). In at least one embodiment, the nonionic surfactants are chosen from polyethoxylated sorbitol esters, in particular polyethoxylated sorbitol monoesters; polycondensates of ethylene oxide and propylene oxide (poloxamers), for instance the products marketed under the trade name PLURONIC by BASF-Wyandotte; condensates of propylene glycol; polyethoxylated hydrogenated castor oil, for instance, cremophors; and sorbitan fatty esters.

Non-limiting examples of amphoteric surfactants include, for example, long chain imidazoline derivatives such as the product marketed under the trade name ‘Miranol C2M’ by Miranol; long chain alkyl betaines, such as the product marketed under the tradename ‘Empigen BB’ by Albright+Wilson, and long chain alkyl amidoalkyl betaines, such as cocamidopropylbetaine, and mixtures thereof.

The oral care composition may include amphoteric surfactants chosen from derivatives of aliphatic secondary and tertiary amines, in which the aliphatic radical can be a straight chain or branched and wherein one of the aliphatic substituents contains from about 8 to about 18 carbon atoms and one contains an anionic water-solubilizing group, such as carboxylate, sulfonate, sulfate, phosphate, or phosphonate. Other exemplary amphoteric surfactants are betaines, such as cocamidopropyl betaine, lauryl dimethyl betaine (sold under the trade name Macat LB), cetyl dimethyl betaine, and cocoamphodiacetate. Additional amphoteric surfactants and nonionic surfactants can be found in U.S. Pat. No. 4,051,234, which is incorporated herein in its entirety for all purposes.

Examples of cationic surfactants that may be present in the oral care composition include cetyl pyridinium chloride, coamidopropyl PG dimonium chloride phosphate (Phospholipid CDM), myristylamidopropyl PG dimonium chloride phosphate (Phospholipid PTM), stearamidopropyl PG dimonium chloride phosphate (Phospholipid SV), steapyrium chloride (Catemol WPC), and other suitable cationic materials. The cationic surfactants may be D,L-2-pyrrolidone-5-carbo-xylic.

The oral care composition may include a zinc source. The zinc source may be a zinc ion source. The zinc source may be present in the oral care composition in an amount from about 0.1 to about 8 wt. %, based on the total weight of the oral care composition. For example, the amount of zinc source present in the oral care composition may be from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.3 to about 8 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %, or any range or subrange thereof, based on the total weight of the oral care composition.

The zinc source may be in the form of a salt. For example, the zinc source may comprise one or more zinc salt(s) chosen from zinc sulfate, zinc chloride, zinc acetate, zinc phenol sulfonate, zinc borate, zinc bromide, zinc nitrate, zinc glycerophosphate, zinc benzoate, zinc carbonate, zinc carnosine, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, zinc lactate, zinc glycinate, zinc phosphate, and a combination of two or more thereof. In some embodiments, the zinc source is selected from zinc citrate, zinc oxide, zinc phosphate, zinc lactate, zinc sulfate, zinc silicate, zinc gluconate, and a combination of two or more thereof.

The oral care composition may include two or more, three or more, four or more, five or more, or six or more zinc sources. For instance, oral care composition may comprise 2 to 7, 2 to 6, 2 to 5, 2 to 5, or 2 to 4; 3 to 7, 3 to 6, 2 to 5, or 3 to 5 zinc sources. In some embodiments, the oral care composition comprises zinc oxide, zinc citrate, zinc phosphate, or a combination of two or more thereof.

The oral care compositions may be formulated to have a weight ratio of the amount of zinc oxide to zinc citrate of from about 1.5:1 to about 4.5:1. For instance, the weight ratio of the amount of zinc oxide to zinc citrate may be from about 1.5:1 to about 4.5:1, about 1.5:1 to about 4:1, about 1.5:1 to about 3.5:1, about 1.5:1 to about 3:1, about 1.5:1 to about 2.5:1; from about 2:1 to about 4.5:1, about 2:1 to about 4:1, about 2:1 to about 3.5:1, about 2:1 to about 3:1; from about 2.5:1 to about 4.5:1, about 2.5:1 to about 4:1, about 2.5:1 to about 3.5:1; from about 3:1 to about 4.5:1, about 3:1 to about 4:1, or any range or subrange formed thereof. In some embodiments, the weight ratio of the amount of zinc oxide to zinc citrate is about 2:1, about 2.5:1, about 3:1, about 3.5:1, about 4:1, or a range formed thereof.

In some embodiments, the oral care compositions may include one or more sweeteners. The oral care composition may include caloric sweeteners and/or non-caloric sweeteners. Examples of non-caloric sweeteners include saccharin, for example, sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 50 wt. %, based on the total weight of the oral care composition. For example, the oral care composition may have a total amount of sweetener(s) from about 0.1 to about 40 wt. %, about 0.1 to about 30 wt. %, about 0.1 to about 20 wt. %, about 0.1 to about 10 wt. %, about 0.1 to about 5 wt. %, about 0.1 to about 3 wt. %; from about 1 to about 50 wt. %, about 1 to about 40 wt. %, about 1 to about 30 wt. %, about 1 to about 20 wt. %, about 1 to about 10 wt. %, about 1 to about 5 wt. %, about 1 to about 3 wt. %; from about 5 to about 50 wt. %, about 5 to about 40 wt. %, about 5 to about 30 wt. %, about 5 to about 20 wt. %, about 5 to about 10 wt. %; from about 10 to about 50 wt. %, about 10 to about 45 wt. %, about 10 to about 40 wt. %, about 10 to about 35 wt. %, about 10 to about 30 wt. %, about 10 to about 25 wt. %; from about 15 to about 50 wt. %, about 15 to about 45 wt. %, about 15 to about 40 wt. %, about 15 to about 37 wt. %, about 15 to about 34 wt. %, about 15 to about 31 wt. %, about 15 to about 28 wt. %, about 15 to about 25 wt. %; from about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 50 wt. %, about 37 to about 45 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less of caloric sweeteners, based on the weight of the oral care composition. In at least one embodiment, the oral care composition contains about 0 wt. % or 0 wt. % of caloric sweeteners, based on the weight of the oral care composition.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, and various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more colorants. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the oral care composition.

The oral care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween, e.g., as measured as a 10 wt. % solution. The pH of the solution (e.g., any of the foregoing pH ranges) may be measured as a 10 wt. % solution, whereby the solution comprises 10 wt. % of the oral care composition with the remainder being water.

In accordance with additional aspects, a method is provided for preparing an oral care composition, the method comprising cross-linking a polyacrylic acid and/or salt thereof to form a cross-linked polyacrylic acid and/or salt thereof; and chelating a stannous ion source with the cross-linked polyacrylic acid and/or salt thereof. The method may include cross-linking the polyacrylic acid and/or salt thereof with any of the polyphosphates, basic amino acids, bicarbonates, carbonates, and/or hydroxides disclosed herein and, e.g., in an amount the amounts disclosed herein. In some instances, the polyacrylic acid and/or salt thereof is cross linked with a polyphosphate, a basic amino acid, or a combination thereof, such as those disclosed above. The step of cross-linking the polyacrylic acid and/or salt thereof may comprise neutralizing the polyacrylic acid and/or salt thereof.

The method includes chelating a stannous ion source with the cross-linked polyacrylic acid and/or salt thereof. In some embodiments, the stannous ion source is mixed with the cross-linked polyacrylic acid and/or salt thereof before the addition of a metal ion source, such as from a fluoride ion source, a surfactant, a potassium source, a nitrate source, and/or a zinc source, including those disclosed herein. For instance, the stannous ion source may be the first ingredient mixed with the cross-linked polyacrylic acid and/or salt thereof.

The method may further include adding one or more additional ingredient (e.g., after mixing and/or chelating the stannous ion source with the cross-linked polyacrylic acid and/or salt thereof) to the cross-linked polyacrylic acid and/or salt thereof. The additional ingredient may be selected from any of the above potassium sources, nitrate sources (e.g., nitrate ion sources), fluoride sources (e.g., fluoride ion sources), polyols, abrasives, surfactants, thickening agents other than a polyacrylic acid and/or salt thereof, zinc sources, sweeteners, flavoring agents, colorants, and/or water.

### EXAMPLES

### Example 1

Two non-limiting example compositions (Ex. A and Ex. B) and two comparative compositions (Comp. 1 and Comp. 2) were all prepared in the form of a dentifrice. The formulation for Example Compositions A and B and Comparative Compositions 1 and 2 are shown in Table 1 (below).

TABLE 1

| | Comp. 1 | Comp. 2 | Ex. A | Ex. B |
|---|---|---|---|---|
| US INCI Name | (wt. %) | (wt. %) | (wt. %) | (wt. %) |
| Stannous Fluoride | 0.45 | 0.45 | 0.45 | 0.45 |
| Tetrasodium | 1.2 | 1.4 | 2.4 | 2.4 |
| Pyrophosphate | | | | |
| Carbopol Polymer | | | 0.5 | 0.5 |
| Carrageenan | 1.5 | 1.4 | 0.3 | 0.3 |
| Sorbitol | 20.3 | 18.9 | 19.6 | 19 |
| Glycerin | 25 | 25 | 25 | 25 |
| Titanium Dioxide | 0.5 | 0.5 | 0.5 | 0.5 |
| Silica | 20 | 20 | 20 | 20 |
| Saccharin | 0.4 | 0.4 | 0.4 | 0.4 |
| Flavor | 1 | 1 | 1 | 1 |
| Sodium Lauryl Sulfate | 2.3 | 2.3 | 2.3 | 2.3 |
| Trisodium Citrate | | 1 | | 1 |
| Water | QS to 100 | QS to 100 | QS to 100 | QS to 100 |
| pH (at 10 wt. % solution) | 7.1 | 7 | 7 | 6.8 |
| Citric Acid | | 0.1 | | 0.1 |

### Example 2

Example Compositions A and B and Comparative Compositions 1 and 2 were evaluated to assess the amount of soluble Tin(II) under accelerated aging conditions. Samples from each of Example Compositions A and B and Comparative Compositions 1 and 2 were kept in an environmentally controlled area at a temperature of 60° C. for 2 weeks. The amount of soluble Tin(II) in each sample was measured before being placed in the environmentally controlled area, after 1 week of residing in the environmentally controlled area, and after two weeks of residing in the environmentally controlled area. A summary of the results from the accelerated aging according to this Example is shown in Table 2 (below).

TABLE 2

| | | Comp. | Comp. | | |
|---|---|---|---|---|---|
| | | 1 | 2 | Ex. A | Ex. B |
| Soluble | Δ from Initial to | −48.3 | −33 | −17.2 | −17.9 |
| Tin II | 2 weeks (%) | | | | |

Additional samples from each of Example Compositions A and B and Comparative Compositions 1 and 2 were collected and kept in an environmentally controlled area at a temperature of 40° C. and at a relative humidity (RH) of 75% for 13 weeks per ICH accelerated aging/stress guidelines. The amount of soluble Tin(II) in each sample was measured before being placed in the environmentally controlled area, after 4 weeks of residing in the environmentally controlled area, after 8 weeks of residing in the environmentally controlled area, and after 13 weeks of residing in the environmentally controlled area. The samples of each composition were sealed in representative toothpaste tubes. A summary of the results from the accelerated aging to assess the stability of soluble Tin(II) at a temperature of 40° C. and at a relative humidity of 75% according to this Example is shown below in Table 3.

TABLE 3

| | | Comp. | Comp. | | |
|---|---|---|---|---|---|
| | | 1 | 2 | Ex. A | Ex. B |
| Soluble Tin | Δ from Initial to 13 | −48.2 | −25.9 | −20.6 | −17.8 |
| II (%) | weeks (%) | | | | |

### Example 3

Example Compositions A and B and Comparative Compositions 1 and 2 were evaluated to assess the amount of soluble tin under different accelerated aging conditions. Specifically, the samples from each of Example Compositions A and B and Comparative Compositions 1 and 2 were kept in an environmentally controlled area at a temperature of 40° C. and a relative humidity (RH) of 75% for 13 weeks. The amount of soluble tin in each sample was measured before being placed in the environmentally controlled area, after 4 weeks of residing in the environmentally controlled area, after 8 weeks of residing in the environmentally controlled area, and after 13 weeks of residing in the environmentally controlled area. A summary of the results from the accelerated aging according to this Example is shown in Table 4.

TABLE 4

| | | Comp. | Comp. | | |
|---|---|---|---|---|---|
| | | 1 | 2 | Ex. A | Ex. B |
| Soluble Tin | Δ from Initial to 13 weeks | −22.2 | −13.8 | −9.4 | −3.3 |
| (%) | (%) | | | | |

Without being limited to any particular theory, it is believed that stannous formulations with a pH of greater than 5 often have high levels of oxidation and precipitation of the soluble tin. It was surprisingly beneficial that Example Compositions A and B maintained high levels of soluble tin and Tin(II).

### Example 4

Three non-limiting example compositions (Exs. C-E) and were all prepared in the form of a dentifrice. The formulation for Example Compositions C-E are shown below in Table 5.

TABLE 5

| | Ex. C | Ex. D | Ex. E |
|---|---|---|---|
| US INCI Name | (wt. %) | (wt. %) | (wt. %) |
| Stannous Fluoride | 0.45 | 0.45 | 0.45 |
| Tetrasodium | 2.5 | 2.5 | 3 |
| Pyrophosphate | | | |
| Carbopol Polymer | 0.5 | 0.5 | 0.65 |
| Carrageenan | 0.25 | 0.25 | 0.25 |
| Potassium Nitrate | 0.5 | 0.5 | 0.5 |
| Sorbitol | 19.4 | 19.4 | 18 |
| Glycerin | 25 | 25 | 25 |
| Titanium Dioxide | 0.5 | 0.5 | 0.5 |
| Silica | 20 | 20 | 20 |
| Saccharin | .4 | .4 | 0.4 |
| Flavor | 1 | 1 | 1 |
| Sodium Lauryl Sulfate | 2.3 | 2.3 | 2.3 |
| Trisodium Citrate | | 1 | |
| Citric Acid | | 0.1 | |
| Water | QS to 100 | QS to 100 | QS to 100 |
| pH (at 10 wt. % solution) | 7.2 | 6.8 | 7.1 |

### Example 5

Example Compositions C-E were evaluated to assess the amount of soluble Tin(II) under accelerated aging conditions. Samples from each of Example Compositions C-E were kept in an environmentally controlled area at a temperature of 60° C. for 2 weeks. The amount of soluble Tin(II) in each sample was measured before being placed in the environmentally controlled area, after 1 week of residing in the environmentally controlled area, and after two weeks of residing in the environmentally controlled area. A summary of the results from the accelerated aging according to this Example is shown below in Table 6.

TABLE 6

| | | Ex. C | Ex. D | Ex. E |
|---|---|---|---|---|
| Soluble | Δ from Initial to | −25 | −21.1 | −23.5 |
| Tin II | 2 weeks (%) | | | |

Additional samples from each of Example Compositions C-E were collected and kept in an environmentally controlled area at a temperature of 40° C. and at a relative humidity (RH) of 75% for 13 weeks. The amount of soluble Tin(II) in each sample was measured before being placed in the environmentally controlled area, after 4 weeks of residing in the environmentally controlled area, after 8 weeks of residing in the environmentally controlled area, and after 13 weeks of residing in the environmentally controlled area. A summary of the results from the accelerated aging to assess the stability of soluble Tin(II) at a temperature of 40° C. and at a relative humidity of 75% according to this Example is shown below in Table 7.

TABLE 7

| | | Ex. C | Ex. D | Ex. E |
|---|---|---|---|---|
| Soluble Tin | Δ from Initial to 13 weeks | −18.8 | −18.2 | −20.6 |
| II (%) | (%) | | | |

### Example 6

Example Compositions C-E were evaluated to assess the amount of soluble tin under different accelerated aging conditions. Specifically, the samples from each of Example Compositions C-E were kept in an environmentally controlled area at a temperature of 40° C. and a relative humidity (RH) of 75% for 13 weeks. The amount of soluble tin in each sample was measured before being placed in the environmentally controlled area, after 4 weeks of residing in the environmentally controlled area, after 8 weeks of residing in the environmentally controlled area, and after 13 weeks of residing in the environmentally controlled area. A summary of the results from the accelerated aging according to this Example is shown in Table 8 (below).

TABLE 8

| | | Ex. C | Ex. D | Ex. E |
|---|---|---|---|---|
| Soluble Tin | Δ from Initial to 13 weeks | −3.1 | −9 | −2.8 |
| (%) | (%) | | | |

What is claimed is:

1. An oral care composition comprising:
a stannous source; and
a cross-linked polyacrylic acid and/or a salt thereof,
wherein the personal care composition has a weight ratio of the stannous source to the cross-linked polyacrylic acid and/or a salt thereof of about 4:1 to about 1:4.

2. The oral care composition according to claim 1 further comprising:
about 10 wt. % of water or more, based on the total weight of the oral care composition.

3. The oral care composition according to claim 1, wherein the cross-linked polyacrylic acid and/or the salt thereof is a branched polyacrylic acid and/or a salt thereof.

4. The oral care composition according to claim 1, wherein the cross-linked polyacrylic acid and/or the salt thereof is cross-linked with allyl sucrose and/or allyl pentaerythritol.

5. The oral care composition according to claim 1, wherein the cross-linked polyacrylic acid and/or the salt thereof contains from about 56 to about 68% w/w carboxylic acid groups.

6. The oral care composition according to claim 1, wherein the cross-linked polyacrylic acid and/or the salt thereof has an average molecular weight of 7×10⁵ to 4×10⁹ Da.

7. The oral care composition according to claim 1, wherein the oral care composition comprises about 2 wt. % or less of natural gums and/or polysaccharides.

8. The oral care composition according to claim 1, wherein the stannous source is selected from stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, and a combination of two or more thereof.

9. The oral care composition according to claim 1 further comprising:
an alkali metal polyphosphate selected from a pyrophosphate, a tripolyphosphate, a tetraphosphate, a hexametaphosphate, and a combination of two or more thereof, wherein the alkali metal polyphosphate is present in an amount from about 0.1 to about 7 wt. %, based on the total weight of the oral care composition.

10. The oral care composition according to claim 1 further comprising:
a basic amino acid in an amount from about 0.1 to about 10 wt. %, based on the total weight of the oral care composition.

11. The oral care composition according to claim 1 further comprising:
a potassium source comprising potassium nitrate, potassium citrate, or a combination thereof, wherein the potassium source is present in an amount from about 0.1 to about 6 wt. %, based on the total weight of the oral care composition.

12. The oral care composition according to claim 1 further comprising:
a polyol comprising glycerin, inositol, maltitol, mannitol, sorbitol, xylitol, propylene glycol, polypropylene glycol (PPG), polyethylene glycol (PEG), a block copolymer of PPG and PEG, a saccharide, or a combination of two or more thereof, wherein the polyol is optionally present in an amount from about 30 to about 68 wt. %, based on the total weight of the oral care composition.

13. The oral care composition according to claim 1 further comprising:
an organic acid buffer system present in an amount from about 0.2 to about 4 wt. %, based on the total weight of the oral care composition.

14. The oral care composition according to claim 13, wherein the organic acid buffer system comprising citric acid and trisodium citrate in a weight ratio of citric acid to sodium citrate of about 1:3 to about 1:8.

15. The oral care composition according to claim 1, wherein the oral care composition has a reduction of less than about 25% of soluble Tin II when maintained at a temperature of 60° C. for 2 weeks.

16. The oral care composition according to claim 1, wherein the oral care composition has a reduction of less than about 25% of soluble Tin II when maintained at a temperature of 40° C. and at a relative humidity of 75% for 13 weeks.

17. The oral care composition according to claim 1, wherein the oral care composition has a reduction of less than about 10% of the total amount of soluble tin when maintained at a temperature of 40° C. and a relative humidity of 75% for 13 weeks.

18. The oral care composition according to claim 1 comprising:
a polyphosphate and/or a salt thereof,
a polyol; and
from about 10 to about 40 wt. % of water,
wherein the oral care composition has a pH of about 5 or more and all weight percentages are based on the total weight of the oral care composition.

19. An oral care composition comprising:
a stannous source;
a polymer system comprising a cross-linked polyacrylic acid, a phosphate/acrylate copolymer, a salt thereof, or a combination thereof; and
a polyphosphate and/or a salt thereof,
wherein the oral care composition has a pH of about 5 or more and all weight percentages are based on the total weight of the oral care composition.

20. A method for preparing an oral care composition comprising:
cross-linking the polyacrylic acid and/or the salt thereof to form a cross-linked polyacrylic acid and/or salt thereof, and
chelating a stannous ion source with the cross-linked polyacrylic acid and/or the salt thereof.
